(19) United States
(12) Patent Application Publication
(10) Pub. No.: US 2017/0165080 A1
(43) Pub. Date: Jun. 15, 2017

(54) OVAL HUMERAL HEADS

(71) Applicant: Zimmer,Inc., Warsaw, IN (US)

(72) Inventor: Andrew Hopkins, Winterthur (CH)

(21) Appl. No.: 15/375,304

(22) Filed: Dec. 12, 2016

Related U.S. Application Data
(60) Provisional application No. 62/266,238, filed on Dec. 11, 2015.

(51) Int. Cl. A61F 2/40

(52) U.S. Cl. CPC A61F 2/4014; A61F 2/4059; A61F 2/4081; A61F 2002/4022; A61F 2002/4051; A61F 2002/4077; A61F 2002/30433

(57) ABSTRACT

A shoulder replacement system including a humeral head having a curved exterior surface and a generally planar exterior surface. The humeral head can define a main body axis extending transversely from the generally planar exterior surface to the curved exterior surface. The humeral head can also include a polar axis extending parallel to the planar exterior surface from an outer edge of the humeral head intersecting the main body axis and an equatorial axis extending parallel to the planar exterior surface from the outer edge of the humeral head intersecting the main body axis and the polar axis. The shoulder replacement system can also include a humeral stem insertable into a humerus and a glenoid implant that can be engaged to bone defining a glenoid cavity and defining an articulating surface.

### CLAIM OF PRIORITY

This patent application claims the benefit of priority, under 35 U.S.C. Section 119(e), to Andrew Hopkins U.S. Patent Application Ser. No. 62/266,238, entitled “OVAL HUMERAL HEADS,” filed on Dec. 11, 2015 (Attorney Docket No. 4394.F34PRV), which is hereby incorporated by reference herein in its entirety.

### TECHNICAL FIELD

This document pertains generally, but not by way of limitation, to an implant system for arthroplasty of shoulders and other joints.

### BACKGROUND

In the natural shoulder joint, the humeral head of the humerus is fitted to the glenoid cavity of the scapula. The shape of the natural humeral head approximates the shape of the glenoid cavity such that the humeral head maintains sufficient contact area between the humeral head and the glenoid cavity as the joint is articulated. Insufficient contact area can result in pain or discomfort during articulating of the joint. Insufficient contact area or an irregularly shaped contact area can cause irregular wear of the glenoid cavity, which can lead to other ailments. The natural glenoid cavity is typically non-spherically shaped making matching the humeral head to the glenoid cavity difficult. The natural glenoid cavity initially can be pear shaped, but can become worn into an oval shape over time. As such, the natural shape of the humeral head can be oval or non-spherically shaped to approximate the shape of the glenoid cavity.

Certain shoulder reconstructive surgeries, such as shoulder semi-arthroplasty, can involve replacing a portion of or an entire damaged humeral head with a humeral implant. The humeral head implant is mounted to the humerus such that the simulated humeral head of the implant approximates the position of the natural humeral head. Following mounting of the humeral implant, the implant humeral head is fitted to the articulating surface of a natural glenoid cavity or an articulating surface of a glenoid implant that approximates the natural shape. The conventional humeral head is typically hemi-spherically shaped, which is easier to manufacture and provide standardized sizes. A drawback of the spherical shape can result in insufficient contact area between the humeral head and the glenoid cavity due to the incompatibility between the hemi-spherical humeral head and the non-spherical articulating surface.

### Overview

The present inventors have recognized, among other things, that a problem to be solved can include insufficient contact area between an implant humeral head improperly fit to an articulating surface of a natural glenoid cavity or a glenoid implant. In an example, the present subject matter can provide a solution to this problem, such as by providing a humeral implant having a non-spherical humeral head shaped to more closely approximate the articulating surface. The humeral head can include a main body having a curved exterior surface and a generally planar exterior surface, the humeral head defining a main body axis extending from the planar exterior surface to the curved exterior surface. The main body can define a polar axis oriented parallel to the generally planar surface and intersecting the main body axis. The main body also can define an equatorial axis transverse to the polar axis and oriented parallel to the generally planar surface, wherein the equatorial axis can intersect the main body axis at the polar axis. In an example, the equatorial axis can differ in length from the polar axis such that the humeral head is non-spherical.

In an example, the main body can define an edge transition between the curved exterior surface and the generally planar exterior surface. The edge transition can be positioned adjacent the outer edge of the main body or positioned inward of the outer edge of the main body such that the curved exterior surface curves inward. In at least one example, the distance of the edge transition from the main body axis along the polar axis can differ from the distance of the edge transition from the main body axis along the equatorial axis such that the curvature of the curved exterior surface vanes around the exterior edge of the main body.

In an example, the main body can define a port for mounting the humeral head to a humeral stem mountable to a humerus. The humeral implant can include an attachment assembly engaging the port to the humeral stem. In at least one example, the port can be offset from the main body axis allowing the humeral head to be positioned offset from the humeral stem and correspondingly the humerus. The offset positioning of the humeral head can improve the contact between the humeral head and the articulating surface.

This overview is intended to provide an overview of subject matter of the present patent application. It is not intended to provide an exclusive or exhaustive explanation of the present subject matter. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a perspective view of a humeral implant according to an example of the present disclosure.

FIG. 2 is a schematic bottom view of a spherical humeral head according to an example of the present disclosure.

FIG. 3 is a side cross-sectional view of the spherical humeral head depicted in FIG. 2.

FIG. 4 is a schematic bottom view of an oval humeral head according to an example of the present disclosure.

FIG. 5 is a side cross-sectional view of the oval humeral head depicted in FIG. 4.

FIG. 6 is a side cross-sectional view of the oval humeral head depicted in FIG. 4.

### DETAILED DESCRIPTION

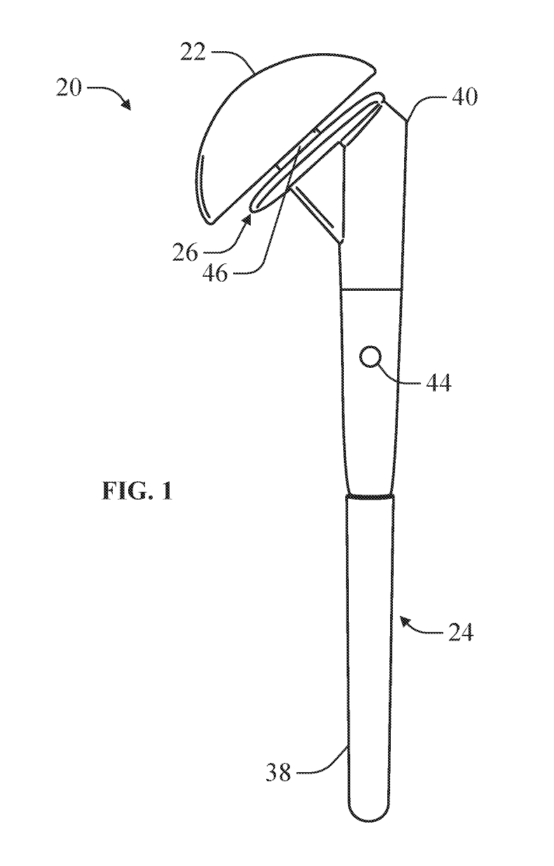

As depicted in FIG. 1, a humeral implant 20, according to an example of the present disclosure, can comprise a humeral head 22 and a humeral stem 24. An attachment assembly 26 can be provided for coupling the humeral head 22 to the humeral stem 24. In operation, the humeral stem 24 is insertable into the humerus to position the humeral head 22 such that the humeral head 22 approximates the natural humeral head. The humeral head 22 can engage the glenoid surface of the glenoid cavity. In an example, a glenoid implant can be mounted to the glenoid cavity to provide a glenoid surface.

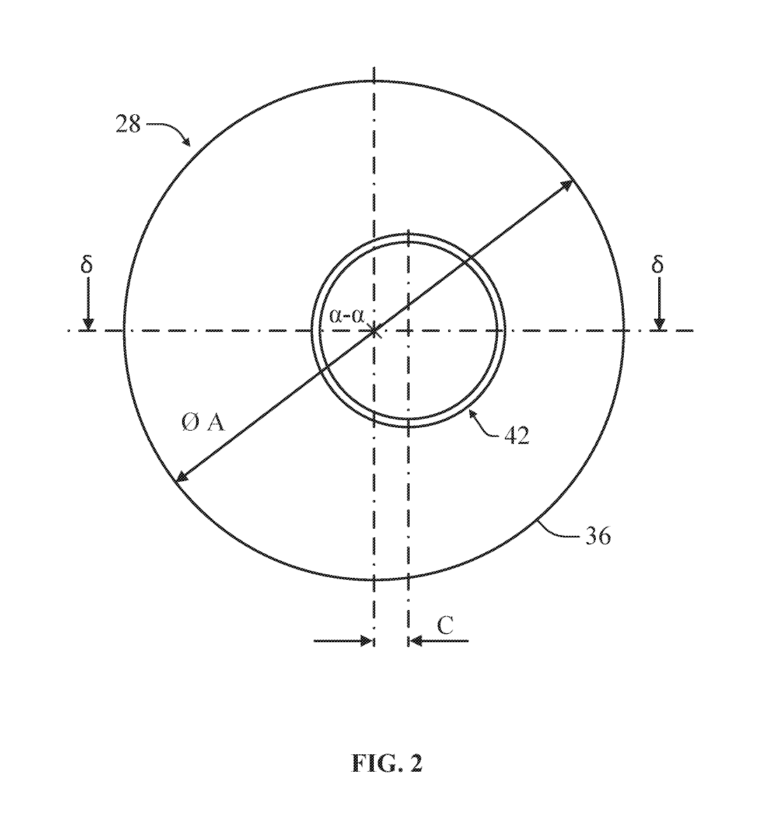

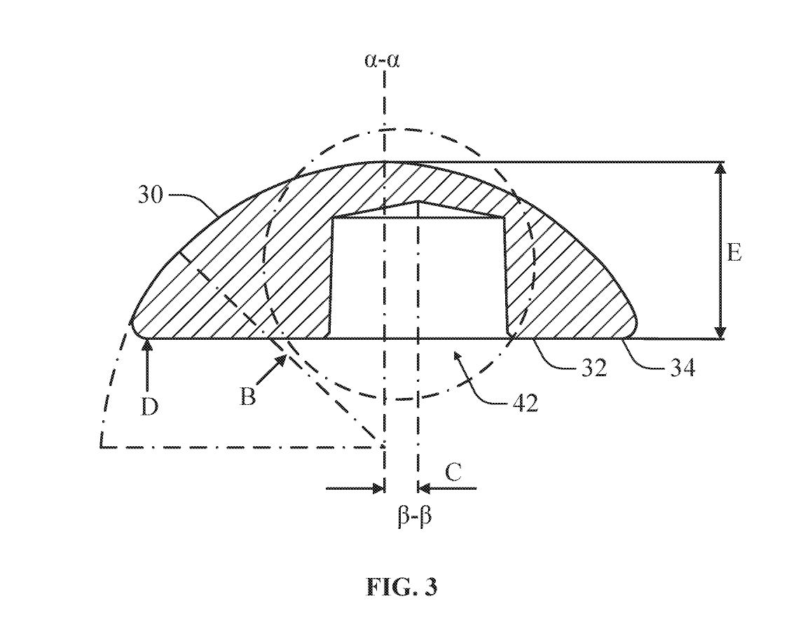

With reference to FIG. 2, in an example, the humeral head 22 can include a main body 28 defining a curved exterior surface 30 and a generally planar exterior surface 32. The main body 28 can further include an edge transition 34 (shown in FIG. 3) positioned between the curved exterior surface 30 and the generally planar exterior surface 32. In an example, the edge transition 34 can be equidistant from the main body axis α-α from an outer edge 36 of the main body 28. In at least one example, the distance of the edge transition 34 from the main body axis α-α is less than the distance from the outer edge 36 from the main body axis α-α. In this configuration, the outer edge 36 is curved as part of the curved exterior surface 30.

With reference again to FIG. 1, in an example, the humeral stem 24 can include a stem portion 38 and a mount portion 40. The stem portion 38 is insertable into humerus after the humeral head is resected. In at least one example, stem portion 38 can have a roughened surface for retaining the stem portion 38 for retaining the stem portion 38 within the humerus. The stem portion 38 can include at least one suture aperture 44 for suturing the stem portion 38 within the humerus. In an example, the mount portion 40 can be angled to the stem portion 38 to angle the humeral head 22 to simulate the natural angle of the humeral head.

As illustrated in FIGS. 1-3, in an example, the mount portion 40 of the humeral stem 24 can include a port 42 for receiving the attachment assembly 26 for attaching the humeral head 22 to the humeral stem 24. The port 42 can define a port axis β-β parallel to the main body central axis α-α. The attachment assembly 26 can include an attachment pin 46 attachable to the generally planar exterior surface 32 such that the attachment pin 46 extends from the generally planar exterior surface 32. The attachment pin 46 can be inserted into the port 42 to attach the humeral head 22 to the humeral stem 24.

As illustrated in FIGS. 2-3, the geometry of a spherically shaped humeral head 22 can be expressed by: a head footprint A; a curvature radius B; a port offset C; an edge transition position D; and a head height E.

As depicted in FIG. 2, the head footprint A can comprise the diameter of the main body 28 intersecting the main body axis α-α. In an example, the diameter of the main body 28 can be measured from opposing sides of the outer edge 36 of the main body 28 through the main body axis α-α along a radial axis δ-δ. In at least one example, the diameter of the main body 28 can be measured from opposing sides of the edge transition 34 of the main body 28 through the main body axis α-α, when the edge transition 34 is at the outer edge 36 of the main body 28.

As depicted in FIG. 3, the curvature radius B can comprise the radial distance from the curved exterior surface 30 to a center point of a sphere having a curvature corresponding to the curved exterior surface 30.

As depicted in FIGS. 2-3, the port offset C can comprise the distance between the main body axis α-α and the port axis β-β when the port 42 is offset from the center of the main body 28. In at least one example, the port 42 can be centered on the main body 28 such that the distance between the main body axis α-α and the port axis β-β is minimal.

As depicted in FIG. 3, the edge transition position D can comprise the distance from the main body axis α-α to the edge transition 34.

As further depicted in FIG. 3, the head height E can comprise the distance from the curved exterior surface 30 to the planer exterior surface 32. In an example, the head height E can comprise the distance between the curved exterior surface 30 and the planar exterior surface 28 at the main body axis α-α. In at least one example, the head height E can comprise the maximum distance between the curved exterior surface 30 and the planar exterior surface 28.

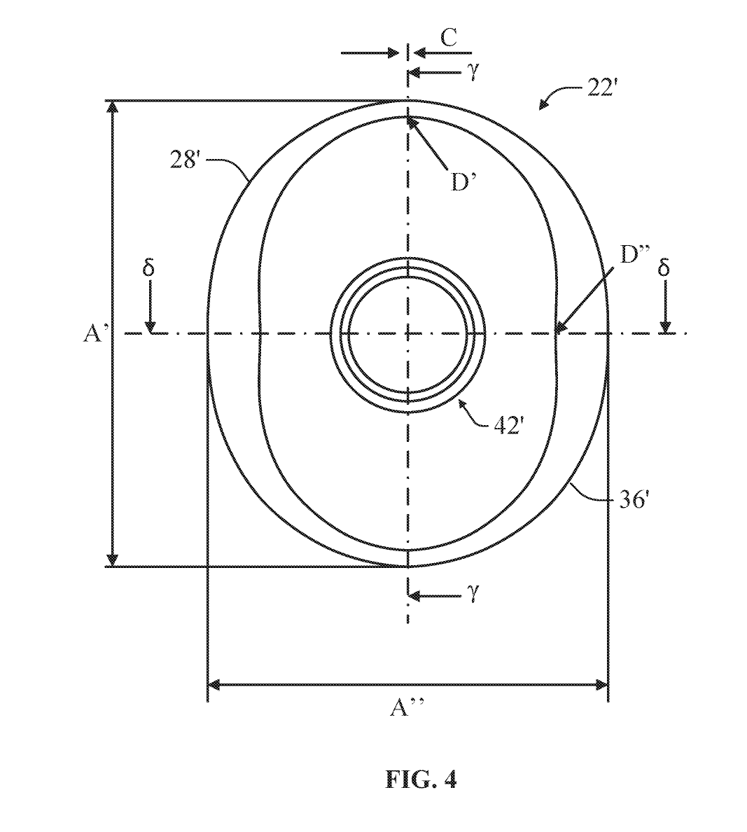

As illustrated in FIGS. 4-6, in an example, the geometry of an oval shaped humeral head 22′ can be expressed by: a polar footprint A′; an equatorial footprint A″; a curvature radius B; a port offset C (not shown); a polar edge transition position D′; an equatorial edge transition position D″; and head height E′.

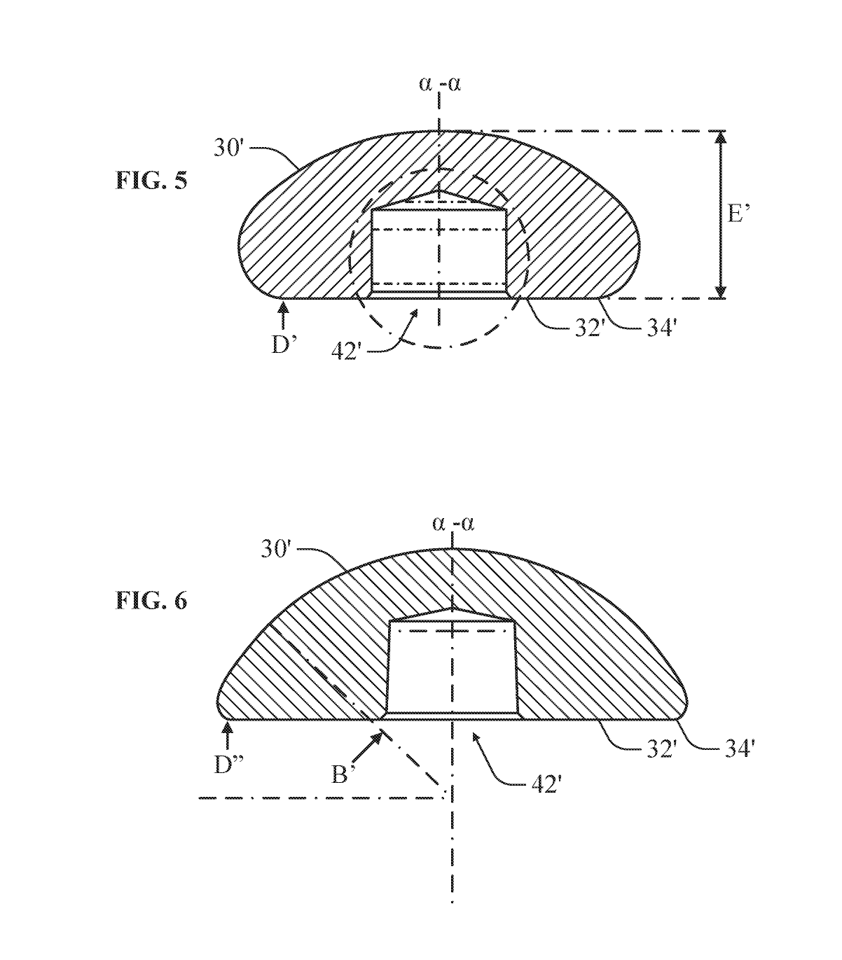

As depicted in FIG. 4, in an example, the humeral head 22 can include a polar axis γ-γ intersecting the main body axis α-α (FIG. 5), wherein the polar axis γ-γ is perpendicular to the main body axis α-α. The humeral head 22 can also include an equatorial axis δ-δ intersecting the main body axis α-α, wherein the equatorial axis δ-δ is perpendicular to the main body axis α-α and the polar axis γ-γ. In this configuration, the polar footprint A′ can comprise the distance from opposing sides of the outer edge 36′ of the main body 28′ through the main body axis α-α and along the polar axis γ-γ. The equatorial footprint A″ can comprise the distance from opposing sides of the outer edge 36′ of the main body 28′ through the main body axis α-α and along the equatorial axis δ-δ. In at least one example, the distance of the main body 28′ for the polar footprint A′ and the equatorial footprint A″ can be measured from opposing sides of the edge transition 34′ of the main body 28′ through the main body axis α-α, when the edge transition 34′ is at the outer edge 36′ of the main body 28′.

As depicted in FIG. 6, the curvature radius B′ can comprise the radial distance from the curved exterior surface 30′ to a center point of a sphere having a curvature corresponding to the curved exterior surface 30′.

As depicted in FIG. 4, the port offset C can comprise the distance between the main body axis α-α and the port axis β-β when the port 42′ is offset from the center of the main body 28′. In at least one example, the port 42′ can be centered on the main body 28′ such that the distance between the main body axis α-α and the port axis β-β is minimal.

As depicted in FIG. 5, the polar edge transition position D′ can comprise the distance from the main body axis α-α to the edge transition 34′ along the polar axis γ-γ. As depicted in FIG. 6, the equatorial edge transition position D″ can comprise the distance from the main body axis α-α to the edge transition 34′ along the equatorial axis δ-δ. In an example, the polar edge transition position D′ can differ from the equatorial edge transition position D″ such that the curvature of the main body 28′ differs around the circumference of the main body 28′.

As depicted in FIG. 5, the head height E′ can comprise the distance from the curved exterior surface 30′ and the planer exterior surface 32′. In an example, the head height E can comprise the distance between the curved exterior surface 30′ and the planar exterior surface 28′ at the main body axis α-α. In at least one example, the head height E can comprise the maximum distance between the curved exterior surface 30′ and the planar exterior surface 28′.

### VARIOUS NOTES & EXAMPLES

Example 1 is a humeral implant, comprising: a humeral head having a curved exterior surface and a generally planar exterior surface, the humeral head defining: a main body axis extending transversely from the generally planar exterior surface to the curved exterior surface, a polar axis extending parallel to the planar exterior surface from an outer edge of the humeral head intersecting the main body axis, and an equatorial axis extending parallel to the planar exterior surface from the outer edge of the humeral head intersecting the main body axis and the polar axis; and a humeral stem insertable into a humerus.

In Example 2, the subject matter of Example 1 optionally includes a port for receiving an attachment assembly for engaging the humeral stem.

In Example 3, the subject matter of Example 2 optionally includes that the port defines a port axis parallel to the main body axis.

In Example 4, the subject matter of any one or more of Examples 2-3 optionally includes that the main body axis is centered in the humeral head, and wherein the port is offset from the main body axis.

In Example 5, the subject matter of any one of the preceding Examples optionally includes an edge transition between the planar exterior surface and the curved exterior surface.

In Example 6, the subject matter of Example 5 optionally includes that the edge transition is positioned at the outer edge of the humeral head.

In Example 7, the subject matter of any one or more of Examples 5-6 includes that the edge transition is positioned inward of the outer edge of the humeral head.

In Example 8, the subject matter of any one or more of Examples 5-7 includes that the edge transition is positioned at the outer edge of the humeral head along the polar axis, and inward of the outer edge of the humeral head at the equatorial axis.

In Example 9, the subject matter of any one of the preceding Examples optionally includes that the humeral head further comprises: a polar diameter defined along the polar axis; and an equatorial diameter defined along the equatorial axis; wherein the polar diameter is different from the equatorial diameter.

In Example 10, the subject matter of Example 9 optionally includes that the polar diameter is greater than the equatorial diameter.

In Example 11, the subject matter of any one of the preceding Examples optionally includes that the humeral stern further comprises: a stem portion insertable into the humerus; and a mount portion for receiving the humeral head.

In Example 12, the subject matter of Example 11 optionally includes that the mount portion is offset from the stem portion such that, upon engagement of the humeral head with the humeral stem, the humeral head is angled relative to the stem portion.

In Example 13, the subject matter of any one or more of Examples 11-12 optionally includes that the stem portion is roughened to improve retention of the stem portion within the humerus.

Example 14 is a shoulder replacement system, comprising: a humeral head having a curved exterior surface and a generally planar exterior surface, the humeral head defining: a main body axis extending transversely from the generally planar exterior surface to the curved exterior surface, a polar axis extending parallel to the planar exterior surface from an outer edge of the humeral head intersecting the main body axis, and an equatorial axis extending parallel to the planar exterior surface from the outer edge of the humeral head intersecting the main body axis and the polar axis; a humeral stem insertable into a humerus; and a glenoid implant engagable to bone defining a glenoid cavity and defining an articulating surface.

In Example 15, the subject matter of Example 14 optionally includes a port for receiving an attachment assembly for engaging the humeral stem.

In Example 16, the subject matter of Example 15 optionally includes that the port defines a port axis parallel to the main body axis.

In Example 17, the subject matter of any one or more of Examples 15-16 includes that the main body axis is centered in the humeral head, and wherein the port is offset from the main body axis.

In Example 18, the subject matter of any one or more of Examples 14-17 includes an edge transition between the planar exterior surface and the curved exterior surface.

In Example 19, the subject matter of Example 18 optionally includes that the edge transition is positioned at the outer edge of the humeral head.

In Example 20, the subject matter of any one or more of Examples 18-19 includes that the edge transition is positioned inward of the outer edge of the humeral head.

In Example 21, the subject matter of any one or more of Examples 18-20 includes that the edge transition is positioned at the outer edge of the humeral head along the polar axis and inward of the outer edge of the humeral head at the equatorial axis.

In Example 22, the subject matter of any one or more of Examples 14-21 includes a polar diameter defined along the polar axis; and an equatorial diameter defined along the equatorial axis; wherein the polar diameter is different from the equatorial diameter.

In Example 23, the subject matter of Example 22 optionally includes that the polar diameter is greater than the equatorial diameter.

In Example 24, the subject matter of any one or more of Examples 14-23 includes a stem portion insertable into the humerus; and a mount portion for receiving the humeral head.

In Example 25, the subject matter of Example 24 optionally includes that the mount portion is offset from the stem portion such that, upon engagement of the humeral head with the humeral stem, the humeral head is angled relative to the stem portion.

In Example 26, the subject matter of any one or more of Examples 24-25 includes that the stem portion is roughened to improve retention of the stem portion within the humerus.

Example 27 is a humeral head for a humeral implant, comprising: a curved exterior surface; and a generally planar exterior surface; wherein the humeral head defines: a main body axis extending transversely from the generally planar exterior surface to the curved exterior surface, a polar axis extending parallel to the planar exterior surface from an outer edge of the humeral head intersecting the main body axis, and an equatorial axis extending parallel to the planar exterior surface from the outer edge of the humeral head intersecting he main body axis and the polar axis.

In Example 28, the subject matter of Example 27 optionally includes a port for receiving an attachment assembly for engaging the humeral stem.

In Example 29, the subject matter of Example 28 optionally includes that the port defines a port axis parallel to the main body axis.

In Example 30, the subject matter of any one or more of Examples 28-29 optionally includes that the port is offset from the main body axis; wherein the main body axis is centered in the humeral head.

In Example 31, the subject matter of any one or more of Examples 27-30 optionally includes that an edge transition between the planar exterior surface and the curved exterior surface.

In Example 32, the subject matter of Example 31 optionally includes that the edge transition is positioned at the outer edge of the humeral head.

In Example 33, the subject matter of any one or more of Examples 31-32. optionally includes that the edge transition is positioned inward of the outer edge of the humeral head.

In Example 34, the subject matter of any one or more of Examples 31-33 optionally includes that the edge transition is positioned at the outer edge of the humeral head along the polar axis inward of the outer edge of the humeral head at the equatorial axis.

In Example 35, the subject matter of any one or more of Examples 27-34 optionally includes that the humeral head further comprises: a polar diameter defined along the polar axis; and an equatorial diameter defined along the equatorial axis; wherein the polar diameter is different from the equatorial diameter.

Example 36, the subject matter of Example 35 optionally includes that the polar diameter is greater than the equatorial diameter.

Each of these non-limiting examples can stand on its own, or can be combined in any permutation or combination with any one or more of the other examples.

The above detailed description includes references to the accompanying drawings, which form a part of the detailed description. The drawings show, by way of illustration, specific embodiments in which the present subject matter can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein,” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the present subject matter should be determined with reference to the appended claims, along with the full scope of equivalents to which such claims are entitled,

What is claimed is:

1. A humeral implant, comprising:
a humeral head having a curved exterior surface and a generally planar exterior surface, the humeral head defining:
a main body axis extending transversely from the generally planar exterior surface to the curved exterior surface,
a polar axis extending parallel to the planar exterior surface from an outer edge of the humeral head intersecting the main body axis, and
an equatorial axis extending parallel to the planar exterior surface from the outer edge of the humeral head intersecting the main body axis and the polar axis; and
a humeral stem insertable into a humerus.

2. The humeral implant of claim 1, the humeral head further comprising:
a port for receiving an attachment assembly for engaging the humeral stem, the port defines a port axis parallel to the main body axis
wherein the main body axis is centered in the humeral head and the port is offset from the main body axis.

3. The humeral implant of claim 1, the humeral head further comprising an edge transition between the planar exterior surface and the curved exterior surface.

4. The humeral implant of claim 3, wherein the edge transition is positioned at the outer edge of the humeral head.

5. The humeral implant of claim 3, wherein the edge transition is positioned inward of the outer edge of the humeral head.

6. The humeral implant of claim 3, wherein the edge transition is positioned at the outer edge of the humeral head along the polar axis, and inward of the outer edge of the humeral head at the equatorial axis.

7. The humeral implant of claim 1, wherein the humeral head further comprises:
a polar diameter defined along the polar axis; and
an equatorial diameter defined along the equatorial axis;
wherein the polar diameter is different from the equatorial diameter;
wherein the polar diameter is greater than the equatorial diameter.

8. The humeral implant of claim 1, wherein the humeral stem further comprises:
a stem portion insertable into the humerus; and
a mount portion for receiving the humeral head.

9. The humeral implant of claim 8, wherein the mount portion is offset from the stem portion such that, upon engagement of the humeral head with the humeral stem, the humeral head is angled relative to the stem portion.

10. The humeral implant of claim 8, wherein the stem portion is roughened to improve retention of the stem portion within the humerus.

11. A shoulder replacement system,comprising:
a humeral head having a curved exterior surface and a generally planar exterior surface, the humeral head defining:
a main body axis extending transversely from the generally planar exterior surface to the curved exterior surface,
a polar axis extending parallel to the planar exterior surface from an outer edge of the humeral head intersecting the main body axis, and
an equatorial axis extending parallel to the planar exterior surface from the outer edge of the humeral head intersecting the main body axis and the polar axis;
a humeral stem insertable into a humerus; and
a glenoid implant engagable to bone defining a glenoid cavity and defining an articulating surface.

12. The shoulder replacement system of claim 11, the humeral head further comprising:
a port for receiving an attachment assembly for engaging the humeral stem, the port defining a port axis parallel to the main body axis
wherein the main body axis is centered in the humeral head and the port is offset from the main body.

13. The shoulder replacement system of any one of claim 11, the humeral head further comprising an edge transition between the planar exterior surface and the curved exterior surface.

14. The shoulder replacement system of claim 13, wherein the edge transition is positioned at the outer edge of the humeral head.

15. The shoulder replacement system of claim 13, wherein the edge transition is positioned inward of the outer edge of the humeral head.

16. The shoulder replacement system of claim 13, wherein the edge transition is positioned at the outer edge of the humeral head along the polar axis and inward of the outer edge of the humeral head at the equatorial axis.

17. The shoulder replacement system of any one of claim 11, wherein the humeral head further comprises:
a polar diameter defined along the polar axis; and
an equatorial diameter defined along the equatorial axis;
wherein the polar diameter is different from the equatorial diameter;
wherein the polar diameter is greater than the equatorial diameter.

18. The shoulder replacement system of any one of claim 11, the humeral stem further comprises:
a stem portion insertable into the humerus; and
a mount portion for receiving the humeral head.

19. The shoulder replacement system of claim 18, wherein the mount portion is offset from the stem portion such that, upon engagement of the humeral head with the humeral stem, the humeral head is angled relative to the stem portion.

20. The shoulder replacement system of claim 18, wherein the stem portion is roughened to improve retention of the stem portion within the humerus.
